(19) United States
(12) Patent Application Publication
(10) Pub. No.: US 2003/0235573 A1
(43) Pub. Date: Dec. 25, 2003

(54) Proteins and methods useful for assessing risk of cardiovascular disease

(75) Inventors: Robert F. Balint, Palo Alto, CA (US); John P. Cooke, Palo Alto, CA (US)

Correspondence Address: FOLEY AND LARDNER, SUITE 500, 3000 K STREET NW, WASHINGTON, DC, 20007, US

(73) Assignee: Unither Pharma, Inc.

(21) Appl. No.: 10/448,328

(22) Filed: May 30, 2003

Related U.S. Application Data
(60) Provisional application No. 60/384,077, filed on May 31, 2002.

(51) Int. Cl. A61K 38/44; C12N 9/06; C12P 21/02; C12N 5/06

(52) U.S. Cl. 424/94.4; 435/69.1; 435/191; 435/320.1; 435/325

(57) ABSTRACT

The invention relates to a mutant protein comprising at least a fragment of a mutant dimethylarginine dimethylaminohydrolase (DDAH) enzyme, wherein the fragment possesses an affinity for asymmetric N,N-dimethyl arginine (ADMA) and/or L,N-monomethylarginine (LNMMA), which exists at lower plasma levels than ADMA, and is deficient in hydrolyzing ADMA or LNMMA to citrulline, releasing citrulline, or both.

CROSS-REFERENCE TO RELATED PATENT APPLICATIONS

This application claims the benefit of U.S. provisional application No. 60/384,077, filed May 31, 2003.

### STATEMENT OF GOVERNMENT RIGHTS

A portion of the work performed during development of this invention utilized U.S. Government funds. The United States Government has certain rights to the invention described herein.

### BACKGROUND OF THE INVENTION

1. Field of the Invention

The invention relates to a mutant protein comprising at least a fragment of a mutant dimethylarginine dimethylaminohydrolase (DDAH) enzyme, where the fragment possesses an affinity for asymmetric N,N-dimethyl arginine (ADMA) and is deficient in hydrolyzing ADMA to citrulline and/or releasing free citrulline. The assay would also be useful to detect L,N-monomethylarginine (LNMMA), which exists at lower plasma levels than does ADMA.

2. Background of the Invention

Atherosclerosis is the major cause of disability in this country and is responsible for 500,000 deaths annually due to coronary artery disease and cerebral vascular attack. Because nitric oxide (NO) has inhibitory effects on many of the key processes that promote atherosclerosis (monocyte adherence, platelet aggregation, vascular smooth muscle proliferation), chronic enhancement of vascular NO production could prove useful in inhibiting or preventing atherogenesis.

Atherosclerosis is accelerated by hyper-cholesterolemia, hypertension, diabetes mellitus, tobacco use, elevated levels of lipoprotein(a) (“Lp(a)”) and homocysteine. All of these disorders are characterized in humans by an endothelial vasodilatory dysfunction well before there is any clinical evidence of atherosclerosis (Cooke and Dzau, 1997). In all of these conditions, the abnormality appears to be due in large part to a perturbation of the nitric oxide synthesis (NOS) pathway. In most of these conditions, the cardiovascular abnormality is reversed or ameliorated by administrating L-arginine, a NO precursor, which NOS metabolizes to produce citrulline and NO (Cooke and Dzau, 1997).

ADMA (asymmetric dimethylarginine) and LNMMA are endogenous competitive antagonists of L-arginine for NOS. LNMMA and ADMA appear to be equipotent as inhibitors of NOS, but LNMMA exists at lower plasma levels than does ADMA. Accordingly, most research efforts have been directed at measurements of ADMA. Elevated levels of ADMA have been found in patients with hypercholesterolemia and atherosclerosis (Bode-Böger et al., 1996; Yu and Xiong, 1994); and plasma ADMA levels are elevated 5-10-fold from normal values in uremic rats and in patients with renal failure (Valiance et al., 1992a,b).

Virtually all risk factors associated with accelerated atherosclerosis are also known to attenuate the synthesis and/or activity of endothelial-derived NO, in association with elevations in ADMA. ADMA may be an important determinant of endothelial vasodilator dysfunction, and potentially, an important new risk-factor for detecting atherosclerosis. Recently, it has been reported that ADMA is an independent predictor of cardiovascular death.

Current methods for detecting ADMA in physiological fluids such as blood, plasma and urine, include extraction, chemical derivatization, isolation by reverse-phase HPLC, IC-mass spectometry, and fluorescence, although other methods may also be available (Chen et al., 1997). However, determination of ADMA or LNMMA in biological specimens is complicated by the presence of two closely related compounds which exhibit similar behavior and/or cross-reactivity in assays, namely, arginine and symmetric dimethylarginine (SDMA). ADMA differs from arginine by only two methyl groups on one of the guanidino nitrogens (see FIG. 1). Further, the concentration of arginine is approximately 100 times that of ADMA in normal human serum. SDMA is more similar to ADMA than arginine, differing only in the position of one methyl group. In normal human serum, the concentration of SDMA is comparable to that of ADMA and is approximately 1 μM.

Thus, there is a need to develop an assay that accurately measures ADMA and LNMMA in biological specimens without interference from cross-reactive substances that compete with ADMA or LNMMA. The accurate measurement of circulating NO synthase inhibitors will be a valuable tool in the early detection and treatment of atherosclerosis and other cardiovascular disorders.

### SUMMARY OF THE INVENTION

The invention relates to a mutant protein comprising at least a fragment of a mutant dimethylarginine dimethylaminohydrolase (DDAH) enzyme, where the fragment possesses an affinity for asymmetric N,N-dimethyl arginine (ADMA) and is deficient in hydrolyzing ADMA to citrulline and/or releasing free citrulline. In some embodiments, the invention has the same characteristics with respect to its affinity for and metabolism of LNMMA. The invention also relates to methods of detecting ADMA and LNMMA in biological specimens and kits for use therewith.

### BRIEF DESCRIPTION OF THE DRAWINGS

FIG. 1 depicts the molecular structures of arginine, asymmetric dimethyl arginine (ADMA) and symmetric dimethyl arginine (SDMA).

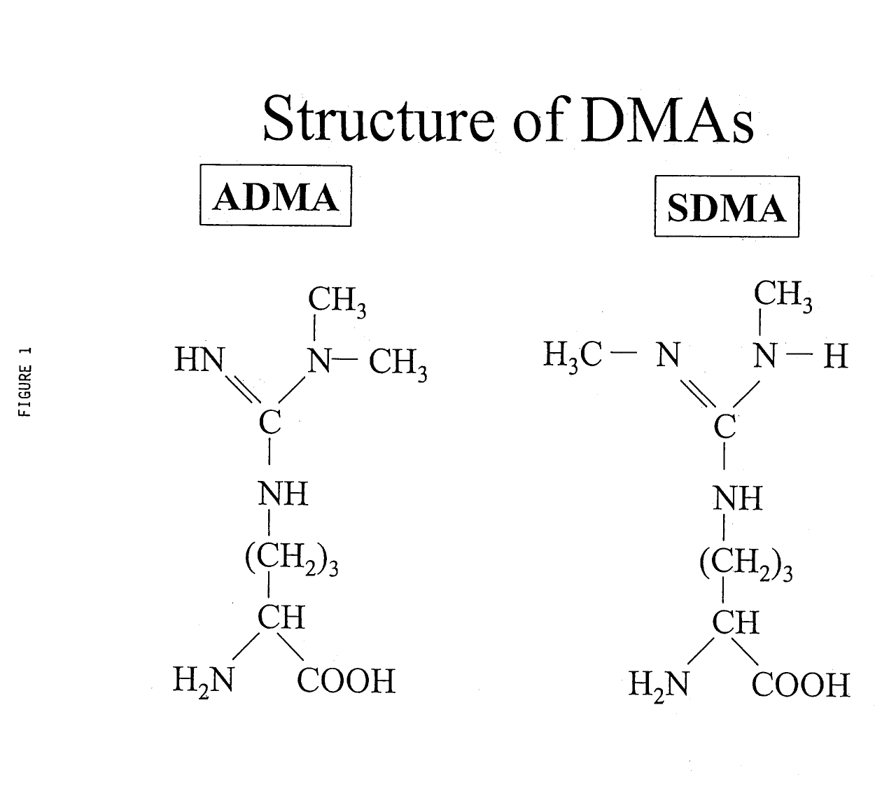

FIG. 2 depicts the relationship between plasma ADMA levels and vascular function (specifically endothelium dependent vasodilation) in human volunteers. Note the inverse relationship between plasma ADMA levels and flow-mediated vasodilation of the brachial artery. The latter response is dependent upon activation of endothelium-derived nitric oxide synthase.

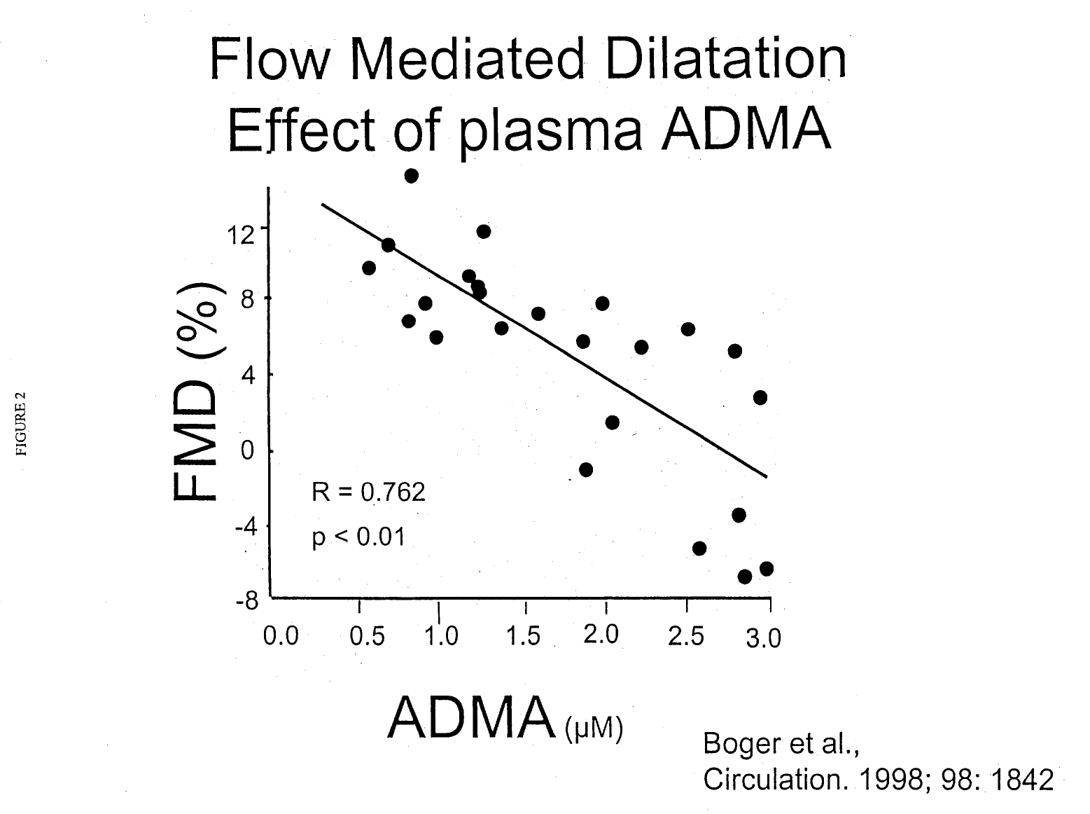

FIG. 3 depicts an original tracing of HPLC measurement of ADMA and LNMMA in human plasma. Homoarginine has been added to the plasma for standardization. Arginine has been removed by first treating the sample with arginase. Normally the arginine peak would occur very close to the homoarginine peak, and often the arginine peak tails into the peaks of LNMMA (labeled as MMA in this diagram), ADMA and SDMA. Note that in this patient, ADMA levels are elevated, because normally, the ADMA and SDMA peaks are similar. SDMA is well separated from ADMA on this tracing, but most of our HPLC records are not so clear due to tailing of the SDMA peak into the ADMA peak.

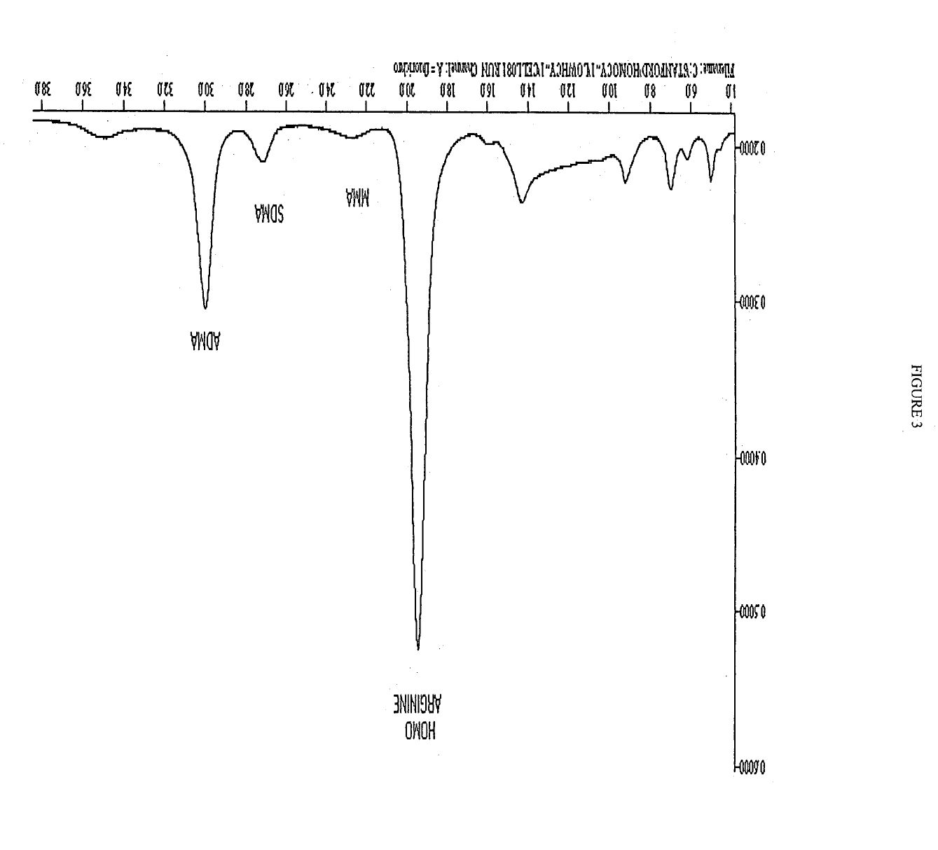

### DETAILED DESCRIPTION OF THE PREFERRED EMBODIMENTS

The invention provides a mutant protein comprising at least a fragment of a mutant dimethylarginine dimethylaminohydrolase (DDAH) enzyme, where the fragment possesses an affinity for asymmetric N,N-dimethyl arginine (ADMA) and is deficient in hydrolyzing ADMA to citrulline and/or releasing free citrulline.

The DDAH enzyme is a zinc(II)-containing enzyme that binds to and hydrolyzes ADMA and/or N-monomethyl arginine (NMMA) to L-citrulline, thus inactivating the inhibitory properties that ADMA and NMMA have towards nitric oxide synthase. Furthermore, the wild-type DDAH enzyme also contains the Cys-His-Glu catalytic triad common to many enzymes. Recently, characterization of DDAH revealed that it is a dimer (Murray-Rust J, et al. Nat Struct Biol. 8(8):679-83 (2001)) having functionally independent catalytic sites.

As used herein, the term protein is used to mean the mature, full-length protein, the unprocessed protein (e.g., containing the signal sequence) or any fragment or portion thereof.

As used herein, a fragment of a protein is used to mean at least 10 contiguous amino acids from any portion or segment from the full-length protein. In one embodiment, the fragment is at least 20 contiguous amino acids. In other embodiments, the fragment is at least 25, 30, 40 or 50 contiguous amino acids. As envisioned in the current context, the fragment may be the entire full-length, mature protein, minus one amino acid. The fragment may also be a part of another protein. It may be fused or bound to another protein or molecule such as an amino acid, a nucleic acid, polynucleotide, a carbohydrate, a lipid, a glycoprotein, a proteoglycan, a polymer or a chemical. The fragment may be part of a fusion protein or may be grafted into another protein. Further, any number of amino acids may be appended to either end of the fragment.

As used herein, the phrase mutant protein means a protein that does not possess all the properties of the wild-type protein or functions to a greater or lesser extent than the wild-type protein. For example, the mutant may be deficient in a particular function, compared to the wild-type, or the mutant may have an increased or enhanced ability or capacity to perform the same function, compared to the wild-type. The product of a mutational event (e.g., a deletion, addition, alteration or substitution etc.) of any number of amino acids in the wild-type DDAH protein or fragment does not necessarily qualify as a mutant of DDAH, as used herein, unless this mutational event is associated with an alteration of the properties or functions of the DDAH or fragment, as described herein. The mutant protein or fragment may be a mutant for more than one property or function. Mutant can also mean that the protein possesses additional or different properties, compared to the wild-type. Furthermore, the mutant need not be the full-length protein, but it can also be a fragment that would, for example, not possess all the properties of the corresponding wild-type fragment or perform the functions to the same extent of the corresponding wild-type fragment. For example, the mutant DDAH fragment of the present invention is deficient in hydrolyzing ADMA to citrulline, compared to the corresponding wild-type fragment of DDAH that is attributed to hydrolyzing ADMA (the catalytic portion or site).

As used herein, “mutant DDAH” can mean the full-length mutant DDAH protein or a fragment of a mutant DDAH protein.

The mutant DDAH enzyme has an amino acid sequence identity that is at least 80% of the sequence of the wild-type enzyme in the domains responsible for binding and metabolizing ADMA or LNMMA. In some embodiments, the mutant DDAH enzyme sequence identity is at least 85% of that of the wild-type enzyme. In some embodiments, the mutant DDAH enzyme sequence identity is at least 90% of that of the wild-type enzyme. In some embodiments, the mutant DDAH enzyme sequence identity is at least 95% of that of the wild-type enzyme. In some embodiments, the mutant DDAH enzyme sequence identity is at least 96% of that of the wild-type enzyme. In some embodiments, the mutant DDAH enzyme sequence identity is at least 97% of that of the wild-type enzyme. In some embodiments, the mutant DDAH enzyme sequence identity is at least 98% of that of the wild-type enzyme. In some embodiments, the mutant DDAH enzyme sequence identity is at least 99% of that of the wild-type enzyme. In some embodiments, the mutant DDAH enzyme sequence identity is 100% of that of the wild-type enzyme.

The sequence identity may be determined by any method. Differences between two amino acid sequences may occur at the C or N terminal positions of the reference amino acid sequence or anywhere between those terminal positions, interspersed either individually among amino acids in the reference sequence or in one or more contiguous groups within the reference sequence.

As a practical matter, whether any particular amino acid molecule is at least a given percentage identical to a reference amino acid sequence refers to a comparison made between two molecules using standard algorithms by hand or machine. Algorithms are well known in the art and can be determined conventionally using publicly available computer programs such as the BLAST family algorithms. (Altschul et al., Nucleic Acids Res. 25:3389-3402, 1997).

The similarity of polypeptide sequences may be examined using the BLASTP algorithm. The BLASTP program is available on the NCBI anonymous FTP server (ftp://ncbi.nlm.nih.gov) under /blast/executables, and from the National Center for Biotechnology Information (NCBI) National Library of Medicine, Building 38A, Room 8N805, Bethesda, Md. 20894, USA. The BLASTP algorithm, may be used to determine polypeptide sequence identity according to the present invention. The BLAST family of algorithms, including BLASTP, is described at NCBI's Internet website at the URL http://www.ncbi.nlm.nih.gov/BLAST/newblast.html and in the publication of The computer algorithm FASTA is available on the Internet at the ftp site ftp://ftp.virginia.edu/pub/fasta/, and from the University of Virginia by contacting David Hudson, Assistance Provost for Research, University of Virginia, PO Box 9025, Charlottesville, Va. Version 2.0u4 [February 1996]. (Pearson and Lipman, Proc. Natl. Acad. Sci. USA 85:2444-2448, 1988; and Pearson, Methods in Enzymol. 183:63-98, 1990.)

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to an inventive polynucleotide preferably have at least one of the following features: (i) they are encoded by genes which contain an open reading frame or partial open reading frame which when translated provide a polypeptide having substantially the same functional properties as inventive polynucleotide; or (ii) they have domains in common.

As used herein, a deficiency in the ability of DDAH to hydrolyze ADMA may be partial or complete. In one embodiment, the mutant DDAH is completely deficient in hydrolyzing ADMA, such that it does not hydrolyze ADMA to any measurable extent. The deficiency can be determined by measuring the levels or concentration of citrulline (using a standard colorimetric assay) in a sample after the sample is exposed to DDAH, or a fragment thereof, in appropriate conditions for enzymatic hydrolysis, whether in vitro, in vivo or in situ. Alternatively, a known amount of ADMA can be added to a solution containing the mutant DDAH, and the decline of ADMA over time measured by HPLC or capillary electropheresis.

Generally, the ability of an enzyme to catalyze a reaction is judged by the Michaelis constant (Kₘ) by methods well-known in the art. A small Kₘ means that the enzyme can achieve half-maximal velocity (of catalyzing the reaction) at low substrate concentrations, indicating an efficient, or quick, enzyme. The higher the Kₘ, the more deficient (i.e., less effective) an enzyme will be in catalyzing a reaction, compared to an enzyme with a lower Kₘ. As an example, if enzyme A, with a Kₘ of 1×10⁻² M, has a higher Kₘ value than enzyme B, with a Kₘ of 1×10⁻⁵ M, enzyme A is 1000 times more deficient than enzyme B in catalyzing the same reaction, under the same conditions (e.g., temperature, pH etc.).

The only two limitations of DDAH as an ADMA sensor are that the binding affinity for SDMA is low, i.e., the K_{M}>100 μM is high and that the products of analyte hydrolysis are released. However, both of these limitations can be mitigated in a single engineering step by one of at least two mechanisms.

By mutagenizing DDAH, displaying the mutant library on the phage surface, and panning against immobilized ADMA in the presence of soluble arginine and SDMA, DDAH mutants with higher specific affinity for ADMA may be obtained. Such variants should have higher affinity by virtue of mutations which increase the affinity for ADMA directly or which prevent the release of the reaction product, citrulline. Mutations which increase substrate affinity typically must do so at the expense of transition state affinity, which reduces the catalytic rate of the enzyme, as desired.

Alternatively, mutations may block product dissociation. Since the reaction intermediate is a covalent thioether adduct on Cys249 of the enzyme via the guanidino carbon of the substrate, water is said to be required to hydrolyze this adduct before the product can be released (Murray-Rust et al., 2001, Nature Structural Biology 8: 679-683). Mutations may therefore either block the access of water to the active site or sterically block diffusion of the product citrulline out of the active site pocket. Either way the analyte would be retained by the enzyme in a way useful for detection, even though the latter type of mutation would not necessarily prevent hydrolysis per se.

To favor the selection of mutant DDAH proteins with high affinity for free ADMA but low (or nonexistent) affinity for arginine or SDMA, it may be helpful to modify the standard panning (forced evolution) strategy. For example, soluble arginine and SDMA may be added to the phage suspensions to inhibit mutant DDAH with high affinities for these compounds from binding to the immobilized ADMA.

The mutant DDAH of the current invention possesses at least some, if not all, of its affinity for ADMA. The mutant DDAH of the current invention may also have increased affinity for ADMA, compared to wild-type DDAH. In one embodiment, the mutant DDAH retains the same affinity for ADMA as the wild-type DDAH. In another embodiment, the mutant DDAH has an increased affinity for ADMA, compared to the wild-type DDAH.

Generally, the affinity of an enzyme (DDAH) towards its substrate (ADMA) can be determined by measuring the Michaelis constant (K_{M}) of the enzyme with respect to the substrate. Methods for measuring K_{M} are well-known in the art. For example, K_{M} can be computed from Lineweaver-Burke plots or Hofstee-Eadie plots as described in any general biochemistry text such as Lehninger or Abeles, Frey, and Jencks. K_{D}, the equilibrium dissociation constant, can be measured by a variety of methods, e.g., competition antibody capture ELISA (Harlow and Lane, Antibodies A Laboratory Manual 1988 Cold Spring Harbor Laboratory Press, Cold Spring Harbor). In this method, for example, haptenized ADMA is immobilized on a solid support and equilibrated with a suspension of labeled or tagged mutant DDAH. Unbound material is washed away, and the bound DDAH is quantified. When this procedure is repeated with increasing amounts of competing ADMA in the suspension, DDAH binding will be reduced by an amount proportional to the ADMA concentration and the K_{D}. When the DDAH concentration is less than or equal to 10% of the K_{D}, the concentration of free ADMA at which the background-corrected signal is reduced by 50% is equal to the K_{D}.

Similar to the K_{D} value, the affinity of an enzyme for its substrate increases as the K_{M} value decreases. The higher the K_{M} value, the lesser the affinity of the enzyme and its substrate towards each other. As an example, if enzyme A, with a K_{M} of 1×10⁻⁴ M, has a higher K_{M} value than enzyme B, with a K_{M} of 1×10⁻⁷ M, enzyme B has an affinity 1000 times greater than that of enzyme A towards the same substrate. In some embodiments, the mutant DDAH has an equilibrium dissociation constant (K_{D}) of no higher than 10⁻⁷M.

The K_{D} is the ratio of the dissociation and association rate constants, k_{D}/k_{A}, respectively. The k_{A} is not likely to ever exceed 10⁴-10⁵M⁻¹sec⁻¹, so under this assumption the k_{D} should be at most 10⁻²-10⁻³sec⁻¹. This is 10⁴-10⁶-fold lower than the probable k_{CAT} of the wild-type enzyme. To achieve this reduction in k_{CAT}, the mutations may either reduce the hydrolysis rate by this amount or reduce the product dissociation rate by this amount or a combination of the two. For example, one mutation may increase the substrate affinity, i.e., reduce the K_{M}, and this would reduce the hydrolysis rate by a factor which is inversely proportional to the effect of the mutation on the substrate k_{A}. That is, if the k_{A} is unaffected, then the hydrolysis rate will be reduced by the same amount as the K_{M}, but if the k_{A} is increased, the hydrolysis rate will be reduced by proportionally lesser amount. Other mutations may inhibit product dissociation, and this effect would be additive with any mutation which decreased the K_{M}.

In one embodiment, the K_{D} of the mutant DDAH, or fragment thereof, and ADMA is at least approximately 1×10⁻⁷M. In another embodiment, the mutant DDAH has a lower affinity towards SDMA than that of the wild-type DDAH.

The mutant DDAH of the present invention may be derived from wild-type DDAH found in animal cells. Preferably, the animal cells are mammalian. More preferably, the animal cells are human.

As used herein, derived is used to mean any technique used to obtain or isolate and purify the mutant DDAH amino acid sequence from the wild-type DDAH amino acid sequence. Examples of derivations include, but are not limited to, site-directed mutagenesis via polymerase chain reaction (PCR) of the DNA encoding the wild-type DDAH and random mutagenesis by using a known chemical mutagen or radiation while DNA replication takes place. The derivation could also utilize standard bacteriophage display techniques (phage display) such as “forced evolution” of related proteins to alter their binding characteristics. Additionally, error-prone PCR may be used for mutagenesis, as described by Cadwell and Joyce in PCR Primer A Laboratory Manual, Dieffenbach and Dveksler, Eds., 1995, Cold Spring Harbor Press, Cold Spring Harbor, N.Y., pp. 583-590. Phage display techniques are described in Phage Display of Peptides and Proteins A Laboratory Manual, Kay, Winter, and McCafferty, Eds., 1996, Academic Press, San Diego. The derivation need not be the result of experimental manipulation but can also be the discovery and subsequent isolation and purification of a naturally occurring mutant of DDAH that has the recited characteristics of the current invention.

By isolated or purified is meant a protein or other molecule being removed from its natural environment. So long as the protein or other molecule is manipulated (or its environment manipulated) such that it is no longer in its natural environment, the purification need not be 100%.

In one embodiment, the mutant proteins of the present invention comprise a detectable label. In another embodiment, the mutant protein and label are conjugated to one another via one or more linking molecules or compounds. Examples of detectable labels, which are well-known in the art, include but are not limited to, enzymes, fluorescers, chemiluminescers, radioisotopes and particles which may be opaque or clear, or may be conjugated with other labels.

The present invention also provides a method for determining the level of ADMA in a biological specimen comprising contacting the biological specimen with mutant DDAH protein, or fragment thereof, and assaying for the amount of ADMA that binds to said mutant protein. In one embodiment, the biological specimen is labeled with a detectable label. In another embodiment, the mutant protein is labeled with a detectable label.

As used herein, a biological specimen can mean any sample, in whole or in part, of a tissue, organ or body fluid. The methods may be employed in an in vivo, in vitro or in situ environment. Examples of specimens include, but are not limited to, whole blood or any fraction thereof, urine, saliva, gastric juices, cerebrospinal fluid, bile, organs, or any portion thereof, blood vessels, or portions thereof, muscle tissue, nerve tissue, and malignant and benign tumor tissue. Further, the specimen also include individual cells and cell cultures which were isolated from a biological sample. Cells and cell cultures include the primary cell culture, as well as any subsequent passages of the primary culture.

Further, the biological sample may be treated, preserved, concentrated, diluted, manipulated (e.g., pH adjusted, buffers added) or altered (e.g., particles precipitated out) prior to the methods of the current invention, depending on the nature of the sample and the nature of the assay. For example, anticlotting factors may be added to whole blood, or alternatively, the red blood cells may be removed, the blood may be citrated or heparinized, etc. The untreated or treated sample may then be combined with the other reagents appropriate for an assay or incubated as appropriate, and then assayed.

Any assay that will accurately measure the relative or absolute levels of ADMA bound to the mutant DDAH protein can be used in the context of the current invention. These assays may be modeled on assays, where ADMA or DDAH is substituted for the available analytes or the binding protein, respectively. The assays include, but are not limited to, spectrophotometric detection, fluorimetric detection, visual detection and detection of radioactivity and may use any of a wide variety of reagents that are commercially available or may be developed along with methodologies described in the literature.

Various protocols for immunoassays may be employed using a variety of reagents. For example, a competition between a mimic of ADMA (a compound which competes with ADMA for binding to the mutant DDAH protein) and any ADMA in the biological specimen, and subsequently measuring the amount of the mutant DDAH protein that binds to the ADMA mimic. This can be achieved by having the ADMA mimic bound to a solid surface, such as, for example a wall of a vessel or a microtiter plate or a particle, which can be separated from the medium.

Channeling may also be used whereby bringing two of the binding proteins together, one obtains a different signal. For example, a fluorescence energy transfer protocol uses a polyepitopic reagent, which has at least two epitopes competitive with ADMA, and two different mutant DDAH proteins, one with a fluorescer, which acts as a donor, and one with a fluorescer, which acts as a receiver. When the two different mutant proteins are bound to the polyepitopic reagent there will be energy transfer, so that by using excitation light to excite the donor, measurable wavelengths of light are emitted.

An antibody directed against the complex formed by the mutant protein and bound ADMA is another approach toward detecting ADMA in the sample. The antibody could then be detected using standard techniques common to ELISA or reverse ELISA techniques. Enzyme-linked immunosorbant assays (ELISA) of one sort or another have been widely used for more than 25 years for the detection and measurement of analytes in bodily fluids (Harlow and Lane, 1988). Their principal advantages stem from the unparalleled affinities and specificities of monoclonal antibodies (mAbs) for analyte tagging, and the unparalleled catalytic power of enzymes for signal amplification. More recently, the immunoassay repertoire has been expanded by the development of homogeneous solution phase assay formats which can be processed in fewer steps than ELISA with improved kinetics and sensitivity (Kopetzki et al., 1994; Coty et al., 1994; Henderson et al., 1986).

In conventional ELISA, haptenized mutant DDAH is immobilized on the surface of microtiter plate wells (Harlow and Lane, 1988). For example, ADMA can be adsorbed out of solution by binding to the immobilized hapten, and after washing, the bound ADMA is detected by binding a secondary antibody-enzyme conjugate which reacts with a chromogenic substrate, producing a signal which is proportional to the amount of bound ADMA.

Fluorescence Polarization Immunassay (FPIA) is another homogeneous solution phase immunoassay for small molecule analytes, which has the unique advantage that a positive signal is generated by competition. FPIA detects the difference between mutant DDAH-bound and free fluorescently-labelled ligand as the polarization of emitted light when excited by plane-polarized light. Small molecule ligands tumble so fast during the excited state that emitted light is nearly isotropic, whereas, the larger mutant DDAH-bound ligand hardly rotates at all during the excited state and therefore emits highly polarized or anisotropic light. Analyte is measured by its ability to compete with label for binding to mutant DDAH, and thereby lower the polarization of emitted light, and increase its intensity at certain angles relative to the incident light. Sensitivity is a function of mutant DDAH affinity and the size difference between bound and free ligand. With nanomolar affinities and a large size difference between free and mutant DDAH-bound analyte, sub-picomolar concentrations may be detectable. ADMA may be conjugated to many fluorophores such as fluorescein by reaction of the free primary amino group of ADMA with an activated derivative of the fluorophore such as fluorescein isothiocyanate (FITC). Free ADMA is measured as a function of its concentration-dependent ability to compete with fluorescein-ADMA for binding to mutant DDAH thereby reducing polarized emission.

Additional immunoassay formats which could be used with our mutant DDAH include, but are not limited to, radioimmunoassay (RIA; Lauritzen et al., 1994), cloned enzyme donor immunoassay (CEDIA; Coty et al., 1994), biomolecular interaction analysis (BIA; Fagerstam et al., 1992), and fluorescence resonance energy transfer immunoassay (FRET; Youn et al., 1995). Like ELISA, most immunoassays for small, monovalent molecules are competitive inhibition assays in which the specimen analyte competes with labeled ligand for binding to mutant DDAH. In RIA the ligand is radioactive. In CEDIA the ligand is conjugated to the α-fragment of β-galactosidase, such that mutant DDAH binding inhibits enzyme activity. Thus, competitive inhibition of the mutant DDAH-ligand interaction by specimen analyte results in an increase in chromogenic enzyme activity. In the FRET immunoassay the ligand is labeled with a fluorophore which can transfer its energy detectably to a fluorophore attached to the mutant DDAH only when both are in close proximity. BIA does not assay by competitive inhibition of binding, but rather directly monitors the interaction of specimen analyte with immobilized mutant DDAH using a phenomenon called surface plasmon resonance, whereby the refractive index at the antibody-bound surface is detectably altered when analyte binds to the mutant DDAH.

If convenient, the assay may employ a fluorescence activated cell sorter and fluorescent particles employed. The assay would provide that the number of fluorescent particles counted would be related to the amount of ADMA present. For example, by having a competitive assay between particles to which ADMA is conjugated and ADMA in the specimen for fluorescently labeled mutant DDAH, the degree to which the particles are labeled with the fluorescent mutant DDAH will be proportional to the amount of ADMA in the specimen. One would then compare the number of fluorescent particles counted with the specimen as compared to a control value.

The following table organizes various assays which may find use in the subject invention.

| Immunoassay type | | Primary quantifier | Example |
|---|---|---|---|
| Com- | Inverse | labeled mutant DDAH in complex | ELISA¹ |
| petitive - | | with competitor | |
| non-linear | | | |
| pro- | | | |
| portionality | | | |
| of analyte to | | labeled competitor in complex with | FPIA² |
| primary | | mutant DDAH | |
| quantifier | | | |
| | Direct | Ratio of unbound labeled competitor | FRET3 |
| | | to bound | |
| | | unbound labeled competitor | CEDIA⁴ |

| | | |
|---|---|---|
| Non-competitive- | Labeled mutant DDAH in complex | APEIA⁵ |
| linear proportionality | with analyte | |
| of analyte to | | |
| primary quantifier | Unlabeled mutant DDAH -analyte | BIA⁶ |
| | complex | |

1. Competitor is the immobilized antigen and bound label is inversely proportional to analyte.
2. Polarization of fluorescent label is inversely proportional to analyte.
3. Donor fluorescence is proportional analyte, optional acceptor fluorescence is inversely proportional analyte.
4. Competitor is enzyme-analyte conjugate. Only free competitor is active and proportional to analyte.
5. Analyte protected enzyme immunoassay; only analyte-bound enzyme-antibody fusion is active. Available from Panorama Research, Inc., Mountain View, CA
6. Bimolecular interference analysis.

The methods of the current invention also include utilizing a molecule, such as an antibody, that binds to the mutant DDAH. The DDAH-specific molecule is bound to a solid surface, such as a microtiter plate, and assaying for the amount of ADMA in a biological sample comprises detecting the amount of labeled mutant DDAH that binds to the surface-bound DDAH-specific molecule.

The present invention also provides for a kit for assaying for the amount of ADMA in a biological specimen comprising the mutant DDAH and at least one reagent. Any reagent useful for detecting the amount of ADMA in a biological sample can be included in the kit.

As used herein, reagents can also include tools (e.g., syringes), supplies (e.g., microtiter plates) or apparatuses useful in performing the assays. Examples of reagents include, but are not limited to, antibodies, antagonists (competitive and non-competitive), agonists, conjugates, colorimetric molecules or solutions, enzymes, secondary messengers, radioisotopes, solutions, syringes, microtiter plates, and lyophilized compounds. In other embodiments, the kit includes an anti-(mutant protein) antibody, and a detectable label.

The antibodies which are employed in the methods and kits of the current invention may be antisera from any convenient source, e.g. bovine, caprine, ovine, canine, equine, rodent, or the like, where the antisera may be purified by selecting out antibodies strongly binding-to the mutant DDAH or the complex formed by mutant DDAH and ADMA. The immunogen for production of the antibodies may be prepared by conjugating the mutant DDAH at an alpha-amino group or the carboxyl group, particularly in the latter case using a linker for bonding to the antigen. Instead of antisera, monoclonal antibodies may be produced in accordance with known ways, including recombinant methods. Particularly, using mice, the mice may be immunized with the DDAH conjugate, splenocytes isolated and immortalized, and then screened for affinity for the mutant DDAH, and perhaps additional characteristics. Clones of interest may be expanded and grown or the genes expressing the anti-DDAH heavy and light chains isolated and manipulated for expression in an appropriate host cell or host. Indeed, as described below, the genes may be mutated to further enhance binding affinity.

### EXAMPLES

1. Isolation of DDAH Mutant With High, Specific Affinity for ADMA.

As the source for mutant DDAH proteins or fragments, one may use a bacteriophage display library of DDAH cloned from endothelial cells (the DDAH 2 isoform) or from neuronal cells (the DDAH 1 isoform). Human DDAH enzyme is amplified from RNA isolated from these cultured cells by reverse transcription and polymerase chain reaction (RT-PCR) using degenerate primers containing the 5′ and 3′ sequences encoding human DDAH. This DDAH-encoding sequence is then ligated into a phagemid vector for expression in the E. coli periplasm as DDAH fused to the amino terminus of the phage minor coat protein (gIIIp). Mutagenesis of the DDAH sequence is then accomplished by Error-Prone PCR as described by Cadwell and Joyce in PCR Primer A Laboratory Manual, Dieffenbach and Dveksler, Eds., 1995, Cold Spring Harbor Press, Cold Spring Harbor, N.Y., pp. 583-590. This procedure can produce an average of ˜1.5 mutations per clone. The mutagenized sequence is ligated back into the phage expression vector, and at least 10 million clones are collected to ensure that all possible two-hit mutants are represented in the library.

Between DDAH and gIIIp in the expression product, three additional elements are encoded by the vector: (1) a 12-residue epitope tag (c-myc) for ELISA detection by anti-epitope antibody, (2) six-histidine tag for purification by affinity chromatography, and (3) a suppressible stop codon (amber) for free DDAH fragment production in non-suppressing hosts without the need for subcloning. The phage library is prepared by quantitative infection of E. coli strain TGI cells (an amber-suppressing host) expressing the DDAH-gIIIp fusions with helper phage, which is essentially wild-type phage containing an antibiotic resistance gene. Phage particles which are produced by the infected cells contain a DDAH mutant on their surface and the DDAH-mutant encoding phagemid inside. The library used is, generally, comprised of phage representing ˜4×10¹⁰ independent DDAH mutant clones.

Phage are panned against immobilized ADMA according to established procedures (McCafferty and Johnson, 1996; McCafferty, 1996). ADMA is conjugated through the alpha-amino group to epsilon-amino groups of exposed lysines on bovine serum albumin (BSA) via a suberate linker, and this conjugate is immobilized on a polystyrene surface. In the panning procedure a suspension containing ˜10¹³ phage particles is exposed to the immobilized ADMA conjugate for 1-2 hours to allow binding equilibration. The suspension should contain about 10 micromolar arginine and 100 nanomolar SDMA to inhibit capture of DDAH fragments with high affinities for these ADMA analogs. These concentrations are selected on the basis of the desired level of discrimination for a mutant DDAH fragment or protein having a desired K_{D} (e.g., 10⁻⁸ M). Bound phage is washed and eluted with triethylamine. From the first round, approximately 6.5×10⁵ phage can be recovered, which are then amplified in E. coli strain TGI back up to ˜10¹³ and subjected to two more rounds of panning, i.e., binding, washing, elution, and amplification. From the final round of panning 1.6×10¹⁰ phage are recovered. An aliquot of this phage population is used to infect E. coli strain HB2151. This strain does not suppress the amber stop codon between DDAH and gIIIp, and therefore the DDAH mutant is expressed in soluble form in the bacterial periplasm without the gIIIp domain.

Free DDAH mutant from clones are screened by ELISA on the immobilized ADMA-suberate-BSA conjugate.

For further characterization and use in clinical assays, DDAH mutants can be purified from the supernatants of large-scale bacterial cultures of the clones. Ultrafiltration is used to increase the concentration approximately 40-fold and replace the bacterial growth medium with phosphate-buffered saline (PBS). DDAH mutants are then affinity-purified using the six-histidine tag at the carboxy terminus of each mutant encoded by the expression vector using immobilized metal ion affinity chromatography (IMAC; Janknecht et al, 1991).

The purified DDAH mutants are then retested for ELISA performance and conditions are optimized. When microtiter wells are coated with 1 microgram ADMA-suberate-BSA, 1×10⁻⁹ M mutant DDAH should give an adequate signal (1-2 OD405 in 30′ with 0.1-0.2 OD background) with anti-myc-tag mouse antibody, horseradish peroxidase-conjugated (HRP) rabbit anti-mouse antibody, and ABTS, a chromogenic substrate for HRP. Under these conditions, i.e., when the DDAH concentration is less than or equal to 10% of the K_{D}, the concentration of free ADMA, at which the background-corrected signal is reduced by 50%, is equal to the K_{D}. When a range of ADMA concentrations (10⁻⁷ M and 10⁻⁸ M) is assayed with 10⁻⁹ M mutant DDAH, at least 50% inhibition may be demonstrated somewhere in the range tested, verifying the original estimates of the K_{D}. Competition ELISA is most sensitive when the analyte concentration is equivalent to the K_{D}, i.e., at 50% inhibition, and such ADMA concentrations correspond to a 10-100-fold dilution of healthy serum.

An additional confirmation of the K_{D} can be obtained by Scatchard analysis of absorbance data. When the ratio of the concentration of the mutant DDAH-ADMA complex to the concentration of free ADMA is plotted against the concentration of the complex, K_{D} may be obtained from the slope (−1/K_{D}). The complex concentration is equal to the total mutant DDAH concentration times (A−A_{b})/(A_{f}−A_{b}), where, A is A405 of the complex whose concentration is to be determined, A_{f} is the A405 of the mutant DDAH in the absence of ADMA, and A_{b} is the A405 of the mutant DDAH in an excess of ADMA. Free ADMA is equal to the total ADMA minus the complex.

2. Determining Serum ADMA Using the Mutant DDAH Fragment in a Competition ELISA

Blood samples from healthy human subjects are employed. After removal of cells from the serum, ADMA is determined by the standard method, which involves removal of serum proteins, fluorescent labeling with o-phthalaldehyde, reversed phase high performance liquid chromatography (HPLC), and post-column, in-line fluorometric detection (Chen et al., 1997). The same specimens are assayed by competition ELISA essentially as described in Example 1 with the mutant DDAH concentration at 1×10⁻⁹ M. A series of dilutions of each specimen ranging from 1:10 to 1:100 is assayed in triplicate, and the results are compared to those of the standard method and a standard curve of pure ADMA from 10⁻⁹ M to 10⁻⁷ M. One may observe excellent agreement between the standard assay and competition ELISA.

3. Optimization of Fluorescence Polarization Immunoassay

ADMA is conjugated through its alpha-amino group to Oregon Green (OG) and purified by reversed phase HPLC. The fluorescence polarization of the free conjugate is determined with a polarizing fluorometer at 498 nm excitation maximum and 524 nm emission maximum over a range of concentrations from 10⁻¹⁰ M to 10⁻⁸ M. The polarization of ADMA-OG should be fairly constant over the entire range with a value of 20±5 at 10⁻⁹ M being typical. At 10⁻¹⁰ M ADMA-OG, polarization is determined after equilibration with various concentrations of mutant DDAH ranging from 10⁻¹⁰ M to 10⁻⁶ M. The minimum value should be equivalent to that of the free conjugate. The maximum value of (150-200) is reached between 10⁻⁷ M and 10⁻⁶ M. The inflection point may thus occur between 10⁻⁷ M and 10⁻⁸ M, consistent with previous estimates of the K_{D}, and this can be confirmed by Scatchard analysis of the fluorescence polarization data. In this case, the ratio of mutant DDAH-ADMA-OG complex to free mutant DDAH is plotted against the complex and, again, the K_{D} is derived from the slope (−1/K_{D}). The complex concentration should be equal to the total ADMA-OG concentration times (P−P_{f})/(P_{b}−P_{f}), where P is the polarization of the complex whose concentration is to be measured, P_{f} is the polarization of free ADMA-OG, and P_{b} is the polarization of ADMA-OG in the presence of an excess of mutant DDAH. Free mutant DDAH is equal to total mutant DDAH minus the complex. Polarization can also be measured in the presence of 10⁻⁷ M free ADMA, which corresponds to a 10-fold dilution of healthy serum, which will diminish the maximum polarization approximately proportionally. Preferably, no detectable inhibition should be observed with 100-fold higher concentration of arginine or with an equivalent concentration of SDMA.

4. Determining Serum ADMA Using the Mutant DDAH Fragment by FPIA

As discussed above, optimum sensitivity of fluorescence polarization to competitive inhibition occurs when the antibody concentration is equivalent to K_{D} and the fluorescent ligand concentration is approximately 10% of the K_{D}. Under these conditions, uninhibited polarization is at the mid-point of its range, and ˜80% inhibition can be achieved with a 100-fold excess of the free analyte. The same specimens analyzed in Example 2 are analyzed again by FPIA with the mutant DDAH concentration at the K_{D}, and the ADMA-OG concentration at 10% K_{D}. A range of dilutions from 1:2 to 1:100 are assayed and compared to a standard curve ranging from 10⁻⁶ M to 10⁻⁸ M. Again, the results may agree with the standard assay as well as with the competition ELISA.

All publications and patent applications mentioned in this specification are herein incorporated by reference to the same extent as if each individual publication or patent application was specifically and individually indicated to be incorporated by reference.

### Literature Cited

Balint R F and Larrick J W (1993) Antibody engineering by parsimonious mutagenesis. Gene 137: 109-118

Balint R F and Plooy I. (1995) Protease-dependent streptomycin sensitivity in E. coli: a method for identifying protease inhibitors. Bio/Technology 13:507-510

Bates P C, Grimble G K, Sparrow M P, Millward D J. Myofibrillar protein turnover. Synthesis of protein-bound 3-methylhistidine, actin, myosin heavy chain and aldolase in rat skeletal muscle in the fed and starved states. Biochem J 1983 Aug. 15;214(2):593-605

Bode-Böger S M, Böger R H, Creutzig A, Tsikas D, Gutzki F M, Alexander K, Frolich J C. L-arginine infusion decreases peripheral arterial resistance and inhibits platelet aggregation in healthy subjects. Clin Sci 1994;87(3):303-10

Bode-Böger S M, Böger R H, Thiele W, Junker W, Frölich J C. Elevated L-arginine/dimethylarginine ratio contributes to enhanced systemic NO production by dietary L-arginine in hypercholesterolemic rabbits. Biochem Biophys Res Comm 1996;219(2):598-603

Böger R H, Bode-Böger S M, Thiele W, Junder W, Alexander K, Frolich J C: Biochemical evidence for impaired nitric oxide synthesis in patients with peripheral arterial occlusive disease. Circulation 1997a; in press

Böger R H, Bode-Böger S M, Szuba A, Tsao P S, Chan J R, Tangphao O, Blaschke T F, Cooke J P: ADMA: A novel risk factor for endothelial dysfunction, its role in hypercholesterolemia. N Engl J Rev 1997b; submitted for review

Burke T, Bolger R, Checovich W, and Lowery R. In: Phage Display of Peptides and Proteins, A Laboratory Manual, Kay B, Winter J, McCafferty J, Eds., Academic Press, San Diego, 1996, pp.305-326

Candipan R C, Wang B-Y, Tsao P S, Cooke J P. Regression or progression: dependency upon vascular nitric oxide activity. Arter, Throm, Vas Bio 1996; 16:44-50

Cayatte A J, Palacino J J, Horten K, Cohen R A. Chronic inhibition of nitric oxide production accelerates neointima formation and impairs endothelial-function in hypercholesterolemic rabbits. Arterioscler Thromb 1994; 14:753-759

Chen B M, Xia L W, Zhao R Q. Determination of N(G),N(G)-dimethylarginine in human plasma by high-performance liquid chromatography. J Chromatogr B Biomed. Sci Appl 1997 May 9;692(2):467-471

Cooke J P, Rossitch E, Andon N, Loscalzo J, Dzau V J: Flow activates an endothelial potassium channel to release an endogenous nitrovasodilator. J Clin Invest 1991a;88: 1663-1671

Cooke J P, Stamler J S, Andon N, Davies P F, Loscalzo J: Flow stimulates endothelial cells to release a nitrovasodiator that is potentiated by reduced thiol. Am J Physiol [Heart Circ Physiol] 1990; 28:H804-H812, 1990

Cooke J P, Andon N A, Girerd X J, Hirsch A T, Creager M A: Arginine restores cholinergic relaxation of hypercholesterolemic rabbit thoracic aorta. Circulation 1991b;83:1057-62

Cooke J P, Singer A H, Tsao P, Zera P, Rowan R A, Billingham M E: Anti-atherogenic effects of L-arginine in the hypercholesterolemic rabbit. J Clin Invest 1992;90: 1168-1172

Cooke J P, Dzau V J: Nitric oxide synthase: Role in the genesis of vascular disease. Annu Rev Med 1997;48:489-509

Coty W A, Loor R, Powell M J, Khanna P L (1994) CEDIA homogeneous immunoassays: current status and future prospects. J Clin Immunoassay 17: 144.150

Cox D A, Vita J A, Treasure C B, Fish R D, Alexander R W, Ganz P, Selwyn A P: Atherosclerosis impairs flow-mediated dilation of coronary arteries in humans. Circulation 1989;80:458-465

Creager M A, Girerd X J, Gallagher S J, Coleman S, Dzau V J, Cooke J P: L-arginine improves endothelium-dependent vasodilation in hypercholesterolemic humans. J Clin Invest 1992;90:1248-1253

Dananberg J, Sider R S, Grekin R J: Sustained hypertension induced by orally administered nitro-L-arginine. Hypertension 1993;21:359-363

den Hartog M, Balint R, Larrick J, deBoer M. Generation of a humanized anti-CD40 MAb for treatment of autoimmune diseases. Keystone Antibody Engineering Meeting, Taos, N.M. (1996)

Drexler H, Zeiher A M, Meinzer K, Just H. Correction of endothelial dysfunction in coronary microcirculation of hypercholesterolemic patients by L-arginine, Lancet 1991; 338: 1546-1550

Fägerstam L G, Frostell-Karlsson Å, Karlsson R, Persson B, Rönnberg I. (1992) J Chromatog 597: 397-410

Gaboury J, Woodinan R C, Granger D N, Reinhardt P, Kubes P. Nitric oxide prevents leukocyte adherence: role of superoxide. Am J of Physio 1993;265(3 Pt 2):H862-7

Garg U C, Hassid A. Nitric oxide-generating vasodilators and 8-bromo-cyclic guanosine monophosphate inhibit mitogenesis and proliferation of cultured rat vascular smooth muscle cells, J Clin Invest 1989;83:1774-1777

Girerd X J, Hirsch A T, Cooke J P, Dzau V J, Creager M A. L-arginine augments endothelium-dependent vasodilation in cholesterol-fed rabbits. Circ Res 1990;67:1301-1308

Harlow E and Lane D Antibodies A Laboratory Manual Cold Spring Harbor Laboratory, Cold Spring Harbor, N.Y., 1988

Henderson D R, Friedman S B, Harris J D, Manning W B, Zoccoli M A. CEDIA, a new homogeneous immunoassay system. (1986) Clin Chem 32: 1637-1641

Hoogenboom H R, Griffiths A D, Johnson K S, Chiswell D J, Hudson P, Winter G. (1991) Nucl. Acids Res. 19:4133-4137

Hoogenboom H R Designing and optimizing library selection strategies for generating high-affinity antibodies. Trends Biotechnol 1997 February; 15(2):62-70

Jacobson R H, Zhang X J, Dubose R F, Matthews B W (1994) Three-dimensional structure of beta-galactosidase from E. coil. Nature 369: 761.

Janknecht R, de Martynoff G, Lou J, Hipskind R A, Nordheim A, Stunnenberg H F. (1991) Rapid and efficient purification of native histidine-tagged protein expressed by recombinant vaccinia virus. Proc. Natl. Acad. Sci. (USA) 88:8972-8976

Kalnins A, Otto K, Ruther U, Muller-Hill B. (1983) Sequence of the lacZ gene of Escherichia coli. EMBO J 2: 593-597

Kay B, Winter J, McCafferty J Phage Display of Peptides and Proteins, A Laboratory Manual Academic Press, San Diego, 1996

Kopetski E, Lehnert K, Buckel P (1994) Enzymes in diagnostics: achievements and possibilities of recombinant DNA technology. Clin, Chem. 40: 688-704

Larrick J W, Truitt K E, Raubitschek A A, Senyk G, Wang J C N (1983) Characterization of human hybridomas secreting antibody to tetanus toxoid. Proc. Natl. Acad. Sci. (USA) 80: 6376

Larrick J W, Graham D, Chenoweth D E, Kunkel S, Fendly B M, Deinhart T. (1986) Murine monoclonals recognizing neutralizing epitopes on human C5a Infet. Immun. 55:1867

Larrick J W, Wallace E F, Coloma M J, Bruderer U, Lang A B, Fry K E. (1993) Therapeutic human antibodies derived from PCR amplification of B cell variable regions. Immunological Reviews 130: 69-85

Larrick J W and Balint, R F Recombinant therapeutic human monoclonal antibodies. In: The Pharmacology of Monoclonal Antibodies. Handbook of Experimental Pharmacology. M. Rosenberg and G. Moore (eds). Academic Press, New York, 1993

Lauritzen E, Flyge H, and Holm A. In: Antibody Techniques. V S Malik and E Lillehoj, Eds., Academic Press, San Diego, 1994, pp. 227-258

Lefer A M, Siegfried M R, Ma X L. Protection of ischemia-reperfusion injury by sydnonimine NO donors via inhibition of neutrophil-endothelium interaction. J of Card Pharm 1993;22 Suppl 7:S27-33

Lerman A, McKinley L, Higano S T, Holmes D R: Oral chronic L-arginine-administration improves coronary endothelial function in humans. JACC 1997;29(2) Liu Q, Dreyfuss G. In vivo and in vitro arginine methylation of RNA-binding proteins. Mol Cell Biol 1995 May; 15(5):2800-2808

MacAllister R J, Parry H, Kimoto M, Ogawa T, Rusell R J, Hodson H, Whitley G S J, Vallance P: Regulation of nitric oxide synthesis by dimethylarginine dimethylaminohydrolase. Br. J. Pharmacol. 1996; 119:1533-1540

Marks J D, Tristem M, Karpas A, and Winter G. 1991a. Oligonucleotide primers for polymerase chain reaction amplification of human immunoglobulin variable genes and design of family-specific oligonucleotide probes. Eur. J. Immunol. 21, 985-991

Marks J D, Hoogenboom H R, Bonnert T P, McCafferty J, Griffiths A D, and Winter G. 1991b. By-passing immunization: Human antibodies from V-gene libraries displayed on phage. J. Mol. Biol. 222, 581-597

Marui N, Offerman M K, Swerlick R, Kunsch C, Rosen C A, Ahmad M, Alexander R W, Medford R M. Vascular cell adhesion molecule-1 (VCAM-I) gene transcription and expression are regulated through an antioxidant-sensitive mechanism in human vascular endothelial cells. J Clin Invest 1993;92: 1866-1874

McCafferty J. Phage Display: Factors Affecting Panning Efficiency. In: Phage Display of Peptides and Proteins, Kay B, Winter J, McCafferty J, eds. Academic Press, San Diego, 1996, pp.261-276

McCafferty J and Johnson K. Construction and screening of antibody display libraries. In: Phage Display of Peptides and Proteins, Kay B, Winter J, McCafferty J, eds. Academic Press, San Diego, 1996, pp.79-112.

Mizobuchi M, Inoue R, Miyaka M, Kakimoto Y. Accelerated protein turnover in the skeletal muscle of dystrophic mice. Biochim Biophys Acta 1985 Nov. 22;843(1-2):78-82

Moffatt B A and Studier F W (1986) J Mol Biol 189: 113-130

Moncada S, Higgs E A: The L-arginine-nitric oxide pathway. N Engl J Med 1993;329:2002-2012

Naruse K, Shimizu K, Muramatsu M, Toki Y, Miyazaki Y, Okumura K, Hashimoto H, Ito T. Prostaglandin H2 does not contribute to impaired endothelium-dependent relaxation and long-term inhibition of nitric oxide synthesis promotes atherosclerosis in hypercholesterolemic rabbit thoracic aorta. Arterioscler Thromb 1994; 14:746-752

Petros A, Bennett D, Vallance P: Effect of nitric oxide synthase inhibitors on hypotension in patients with septic shock. Lancet 1991;338:1557

Radomski M W, Palmer R M J, Moncada S. Comparative pharmacology of endothelium-derived relaxing factor, nitric oxide, and prostacyclin in platelets. Br J Pharmacol 1987;92:181-187

Rees D D, Cellek S, Palmer R M J, Moncada S: Dexamethasone prevents the induction by endotoxin of a nitric oxide synthase and the associated effects on vascular tone: an insight into endotoxin shock. Biochem and Biophys Res Corn 1990; 173:541-547

Sambrook J, Frisch E F, Maniatis T, Molecular Cloning A Laboratory Manual 2nd Ed. Cold Spring Harbor Laboratory, Cold Spring Harbor, N.Y., 1189

Schier R, Balint R F, McCall A, Apell G, Larrick J W, Marks J D. (1996) Identification of functional and structural amino-acid residues by parsimonious mutagenesis Gene 169: 147-155

Scott J K and Smith G P (1990) Searching for peptide ligands with an epitope library. Science 249: 386-390

Shesely E G, Maeda N, Kinm H S, Desai K M, Krege J H, Laubach V E, Sherman P A, Sessa W C, Smithies O: Elevated blood pressures in mice lacking endothelial nitric oxide synthase. Proc Nat Acad Sci USA 1996;93(23):13176-81

Short J et al. (1988) Nucleic Acids Res. 16: 7583-7600

Sparks A B, Adey N B, Cwirla S, Kay B K Screening phage-displayed random peptide libraries. In: Phage Display of Peptides and Proteins, Kay B, Winter J, McCafferty J, eds. Academic Press, San Diego, 1996, pp. 227-254

Stamler J S, Mendelsohn M E, Amarante P, Smick D, Andon N, Davies P F, Cooke J P, Loscalzo J: N-acetylcysteine potentiates platelet inhibition by endothelium-derived relaxing factor. Circ Res 1989;65:789-795

Theilmeier G, Zalpour C, Ma A, Anderson B, Wang B-Y, Wolf A, Candipan R C, Tsao P S, Cooke J P. Adhesiveness of mononuclear cells in hypercholesterolemic humans is normalized by dietary arginine. Arter, Throm, Vas Bio (submitted)

Tojo A, Welch W J, Bremer V, Kimoto M, Kimura K, Omata M, Ogawa T, Valiance P, Wilcox C S. Colocalization of demethylating enzymes and NOS and functional effects of methylarginines in rat kidney. Kidney Int 1997 December;52(6): 1593-1601

Tsao P, McEvoy L M, Drexler H, Butcher E C, Cooke J P: Enhanced endothelial adhesiveness in hypercholesterolemia is attenuated by L-arginine. Circulation 1994;89:2176-2182

Tsao P S, Lewis N, Alpert S, Cooke J P. Exposure to shear stress alters endothelial adhesiveness: Role of nitric oxide. Circulation 1995;92:3513-3519

Tsao P, Buitrage R, Chan J S, Cooke J P. Fluid flow inhibits endothelial adhesiveness: NO and transcriptional regulation of VCAM-1. Circulation (in press)

Vallance P, Leone A, Calver A, Collier J, Moncada S: Endogenous dimethylarginine as an inhibitor of nitric oxide synthesis. J Cardiovasc Pharmacol 1992a;20(Suppl. 12):S60-S62

Vallance P, Leone A, Calver A, Collier J, Moncada S. Accumulation of an endogenous inhibitor of nitric oxide synthesis in chronic renal failure. Lancet 1992b;339(8793):572-5

von der Leyen H E, Gibbons G H, Morishita R, Lewis N P, Zhang L, Nakajima M, Kaneda Y, Cooke J P, Dzau V J: Gene therapy inhibiting neointimal vascular lesion: In vivo transfer of endothelial cell nitric oxide synthase gene. Proc Natl Acad Sci USA 1995;92: 1137-41

Wang B-Y, Singer A, Tsao P, Drexler H, Kosek J, Cooke J P: Dietary arginine prevents atherogenesis in the coronary artery of the hypercholesterolemic rabbit. J Am Coll Cardiol 1994;23;452-58

Winter G, Griffiths A D, Hawkins R E, Hoogenboom H R (1994) Making antibodies by phage display technology. Ann. Rev. Immunol, 12: 433-456

Wolfe A, Theilmeier G, Zalpour C, Ma A, Anderson B, Wang B-Y, Candipan R C, Tsao P S, Cooke J P: Platelet hyperaggregability in hypercholesterolemic humans: Reversal by dietary L-arginine. Annals of Int Med (under review) 1995

Youn H J, Terpetschnig E, Szmacinski H, Lakowicz J R Fluorescence energy transfer immunoassay based on a long-lifetime luminescent metal-ligand complex. Anal Biochem 1995 Nov. 20;232(i):24-30

Yu X, Li Y, Xiong Y: Increase of an endogenous inhibitor of nitric oxide synthesis in serum of high cholesterol fed rabbits. Life Sci. 1994;54:753-758

Zeiher A H, Drexler H, Wollschl ger H, Saurbier B, Just H: Coronary vasomotion in response to sympathetic stimulation in humans: Importance of the functional integrity of the endothelium. J Am Coll Cardiol 1989; 14:1181-1190

What is claimed is:

1. An isolated or purified mutant protein comprising at least a fragment of a mutant dimethylarginine dimethylaminohydrolase (DDAH) enzyme, wherein said fragment possesses an affinity for asymmetric N,N-dimethyl arginine (ADMA), and wherein said mutant protein is deficient in hydrolyzing said ADMA to citrulline and/or releasing said citrulline, or both.

2. The mutant protein of claim 1, wherein said mutant DDAH enzyme possesses an affinity for asymmetric L,N-monomethyl arginine (LNMMA), and wherein said mutant protein is deficient in either hydrolyzing said LNMMA to citrulline or releasing said citrulline, or both.

3. The mutant protein of claim 1, wherein said fragment possesses an increased affinity for ADMA compared to wild-type DDAH.

4. The mutant protein of claim 1, wherein said mutant DDAH is derived from wild-type DDAH from animal cells.

5. The mutant protein of claim 4, wherein said animal cells are mammalian.

6. The mutant protein of claim 5, wherein said mammalian cells are human.

7. The mutant protein of claim 1, wherein said affinity for ADMA has a K_{A} of at least about 1×10⁷ M⁻¹, or a K_{D} of no more than 10⁻⁷ M.

8. The mutant protein of claim 1, further comprising a detectable label.

9. The mutant protein of claim 8, wherein said detectable label is a light absorber, fluorescer, chemiluminescer, enzyme, or radioisotope.

10. A method for determining the level of ADMA in a biological specimen comprising contacting said biological specimen with a mutant protein and assaying for the amount of ADMA that binds to said mutant protein, wherein the mutant protein is an isolated or purified mutant protein comprising at least a fragment of a mutant dimethylarginine dimethylaminohydrolase (DDAH) enzyme, wherein said fragment possesses an affinity for asymmetric N,N-dimethyl arginine (ADMA), and wherein said mutant protein is deficient in hydrolyzing said ADMA to citrulline and/or releasing said citrulline, or both.

11. The method of claim 10, wherein said ADMA in said biological specimen is labeled with a detectable label.

12. The method of claim 11, wherein said detectable label is a light absorber, fluorescer, chemiluminescer, enzyme, or radioisotope.

13. The method of claim 11, wherein said assaying for the amount of ADMA that binds to said mutant protein comprises a fluorescent immunoassay.

14. The method of claim 11, wherein said assaying for the amount of ADMA that binds to said mutant protein comprises an enzyme immunoassay.

15. The method of claim 10, wherein said mutant protein is labeled with a detectable label.

16. The method of claim 15, wherein said detectable label is a light absorber, fluorescer, chemiluminescer, enzyme, or radioisotope.

17. The method of claim 15, wherein said assaying for the amount of ADMA that binds to said mutant protein comprises a fluorescent immunoassay.

18. The method of claim 15, wherein said assaying for the amount of ADMA that binds to said protein comprises an enzyme immunoassay.

19. A method for determining the level of ADMA in a biological specimen comprising contacting said biological specimen with the mutant protein of claim 7 and assaying for the amount of ADMA that binds to said mutant protein.

20. The method of claim 19, wherein said mutant protein is bound to a detectable label selected from the group consisting of fluorescers and enzymes.

21. The method of claim 20, wherein a molecule that binds to said mutant protein is bound to a solid surface and said assaying for the amount of ADMA that binds to said mutant protein comprises detecting the amount of said label bound to said surface.

22. The method of claim 10, wherein said mutant DDAH enzyme possesses an affinity for asymmetric L,N-monomethyl arginine (LNMMA), and wherein said mutant protein is deficient in either hydrolyzing said LNMMA to citrulline or releasing said citrulline, or both.

23. A kit for assaying for the amount of ADMA in a biological specimen comprising a mutant protein and at least one reagent, wherein the mutant protein is an isolated or purified mutant protein comprising at least a fragment of a mutant dimethylarginine dimethylaminohydrolase (DDAH) enzyme, wherein said fragment possesses an affinity for asymmetric N,N-dimethyl arginine (ADMA), and wherein said mutant protein is deficient in hydrolyzing said ADMA to citrulline and/or releasing said citrulline, or both.

24. The kit of claim 23, further comprising an anti-(mutant protein) antibody.

25. The kit of claim 23, further comprising a detectable label.

26. The kit of claim 25, wherein said label is selected from the group consisting of fluorescers and enzymes.
